(19) United States
(12) Patent Application Publication
(10) Pub. No.: US 2024/0390563 A1
(43) Pub. Date: Nov. 28, 2024

(54) METHOD FOR OPERATING A DIALYSIS SYSTEM

(71) Applicant: B. Braun Avitum AG, Melsungen (DE)

(72) Inventors: Stephan Krietemeyer, Melsungen (DE); Philipp Odernheimer, Melsungen (DE); Nino Lieb, Melsungen (DE); Jonas Giesa, Melsungen (DE)

(21) Appl. No.: 18/665,852

(22) Filed: May 16, 2024

(30) Foreign Application Priority Data
May 24, 2023 (DE) 10 2023 113 669.0

(51) Int. Cl. A61M 1/16; G16H 20/40; G16H 40/63

(52) U.S. Cl. CPC A61M 1/1601; G16H 20/40; G16H 40/63

(57) ABSTRACT

A dialysis system has a plurality of hydraulically connected components and a control device. A method of operating the dialysis system includes the steps of receiving, by the control device, component operating data from at least one of the components, the component operating data including at least demand data indicating an expected resource consumption of the component for an upcoming task to be performed by the component, and determining, by the control device, operating parameters for coordinated operation of the components such that an expected resource consumption of the dialysis system is optimized. The step of determining the operating parameters includes determining at least one operating parameter for one or more of the components based on the component operating data and resource availability data and/or task prioritization data.

### CROSS-REFERENCE TO RELATED APPLICATION

This application claims priority under 35 U.S.C. § 119 to German Application No. 10 2023 113 669.0, filed on May 24, 2023, the content of which is incorporated by reference herein in its entirety.

### FIELD

The present disclosure relates to a method of operating a dialysis system, a system comprising a dialysis system, a computer program product and a computer-readable medium.

### BACKGROUND

Until now, various components of dialysis systems, also referred to here as medical devices, have only been hydraulically connected to each other and are programmed individually and usually manually.

As a result, synergies can remain unused, which can lead to a waste of resources, namely water, energy, and/or time.

One task underlying the present disclosure is therefore to provide a method for operating a dialysis system which makes it possible to reduce wastage of resources.

### SUMMARY

The present disclosure relates to a method of operating a dialysis system comprising a plurality of hydraulically connected components and a control device. The method comprises receiving, by the control device, component operating data from at least one of the components, wherein the component operating data comprises at least demand data indicating an expected resource consumption of the component for an upcoming task to be performed by the component. The method also comprises determining, by the control device, operating parameters for coordinated operation of the components such that an expected resource consumption of the dialysis system is optimized. Determining the operating parameters comprises determining, based on the component operating data and based on resource availability data and/or task prioritization data, at least one operating parameter for one or more of the components.

In other words, the component(s) can provide the control device with the component operating data, which includes demand data. This means that the component(s) can register their requirements with the control device. Optionally, the component(s) can report the operating mode and operating phase to the control device. The component operating data, in particular demand data, can be regarded as dynamic data, for example situation-specific data, which is provided by the component(s). The method of the present disclosure thus enables particularly precise optimization results, since it is not necessary to rely exclusively on data stored somewhere, for example from a database, which is specific neither to the situation at hand nor to the actual component at that time. Resource consumption can thus also be reduced particularly effectively.

The dialysis system can be part of a dialysis center or a dialysis practice, for example.

The components, also known as medical devices, can include dialysis machines, hot cleaning devices, reverse osmosis devices, concentrate mixing systems or similar.

The fact that the components are hydraulically connected means that fluid can be transported, for example pumped, between the components. The hydraulic connection can take the form of tubes and/or pipes. The hydraulic connection can be direct or indirect. The dialysis system may include valves that can be used, for example, to adjust a flow through the dialysis system, such as automatically controllable valves.

The control device can be integral with one of the components or separate from the components.

The control device can be configured to receive data from the components by means of a data connection, in particular a bi-directional data connection, with the respective component. The data connection can be wireless or wired.

The term component operating data is to be understood broadly and may include, for example, data relating to the operation of a component, such as data representing the current and/or planned settings of operating parameters, in particular comprising data representing a current and/or planned operating mode of the component and/or the dialysis system, and/or data representing the current state of the component, for example on-state, off-state, or error state. The component operating data may also comprise an expected result, for example an expected output.

As seen above, the component operating data comprises at least demand data indicating an expected resource consumption of the component for an upcoming task to be performed by the component. In particular, the component operating data may indicate the task to be performed and the associated expected resource consumption.

The demand data can indicate an expected resource consumption for one or more resources, for example water consumption and energy consumption. The demand data does not have to indicate the expected resource consumption in a specific representation, for example specific units. It is sufficient for the demand data to be configured in such a way that the resource consumption can be derived from the demand data, for example by the control device.

The control device uses the component operating data to determine operating parameters for coordinated operation of the components. At least one operating parameter is determined for one or more of the components.

The term “operating parameter” is to be understood broadly and can include all parameters whose value affects the operation of a component. For example, changing the value of one or more operating parameters may change the settings of the component. The operating parameters may include, for example, one or more operating modes of the component and/or the dialysis system. Operating parameters may alternatively or additionally comprise, for example, a selection and/or a sequence of tasks to be executed by the component, an execution time for one or more tasks to be executed by the component, and/or settings of the component, in particular at specific times.

Coordinated operation can include the coordinated execution of various tasks to be performed by the components, in particular in a specific sequence and/or at specific times and/or with specific component settings.

The operating parameters are determined in such a way that the expected resource consumption of the (in particular the entire) dialysis system is optimized. Known optimization methods can be used for the optimization. The expected resource consumption of the dialysis system can, for example, be the cumulative resource consumption for a predetermined period of time and/or for a predetermined part of the operation, for example in the form of a group of tasks. The expected resource consumption that is optimized may comprise the consumption of only one resource or the consumption of several resources.

The optimization can be carried out using boundary conditions, for example boundary conditions that ensure safe operation and/or low-wear operation and/or efficient operation of the component and/or reduce the number of on/off cycles.

As seen above, the operating parameter(s) is/are determined based on the component operating data and based on resource availability data and/or task prioritization data. In particular, the operating parameter(s) is/are determined based on the demand data and based on resource availability data and/or task prioritization data. For example, the task prioritization data can be used to determine a relative priority for the various upcoming tasks. The resource availability data can be used to determine the availability of one or more resources, in particular as a function of time. The demand data and the resource availability data can be used to check at which times certain tasks with their associated demand should ideally be carried out so that the available resources can be used optimally, and sufficient resources are available.

In particular, this can reduce the waste of available resources or the additional provision of resources.

In addition, tasks can be carried out efficiently in a simple manner. For example, although a task that a component performs can generally be carried out in a (larger) range of temperatures, it can be carried out particularly efficiently in a sub-range of temperatures. To provide the temperature for this task, the heat emitted by another component when performing a task can be used as a resource. The tasks of the two components can be coordinated in such a way that the task of one component is carried out when the heat emitted by the other component enables it to be carried out in the said sub-range. Otherwise, the task of one component would be carried out with lower efficiency or with additional heating power applied specifically for this purpose. Coordinated operation can therefore, as seen above, increase the overall efficiency of the dialysis system and thus optimize the expected resource consumption.

According to the present disclosure, determining the operating parameters may comprise a grouping of requirements of the components and/or a prioritizing of the tasks to be performed.

Requirements can, for example, include demand data or be derived from demand data, in particular include resource consumption or be derived from resource consumption. Requirements can be grouped or, in other words, assigned to groups. For example, requirements from different components that relate to the same resource can be grouped together. A cumulative, in particular system-wide, requirement can optionally be determined for the respective group, for example a cumulative requirement for a specific resource. The grouping of the requirements can be used to determine the operating parameters, in particular to define a resource distribution to the components.

Prioritizing may include prioritizing the upcoming tasks to be performed by the components based on the task prioritization data, for example, assigning a priority to each of them. To this end, the method may involve the control device determining the upcoming tasks to be performed by the components, for example using the component operating data. Prioritizing the tasks enables available resources to be used efficiently without increasing the risk of critical processes being undersupplied.

According to the present disclosure, determining the operating parameters may comprise determining a timing of the execution of the tasks. In other words, the operation of the components may be coordinated in time.

In particular, a sequence for performing the tasks can be determined and corresponding operating parameters can be selected. For example, a fixed time or a fixed interval for the execution of each task can be determined and defined as operating parameters. Alternatively or additionally, when determining the operating parameters, the operating parameters can be selected in such a way that the completion of one task triggers the start of another task and/or that different tasks are started with a certain time offset.

This ensures system-wide coordinated operation that makes particularly effective use of resources.

According to the present disclosure, the determination of the operating parameters may be carried out taking into account resource availability, in particular taking into account energy availability and/or fluid availability.

The resource availability can be comprised in the resource availability data or derived from resource availability data. In particular, the control device can receive the resource availability data from one or more components of the dialysis system that provide resources, for example storage tanks, water treatment component or heating components.

The operating parameters can be determined in such a way as to optimize how available resources are used. In particular, the optimization can be aimed at using the resources as quickly or as completely as possible. In turn, the use depends on the upcoming tasks and the corresponding expected resource consumption and, if applicable, prioritization of the tasks.

For example, when fluid of a certain temperature is available, tasks that require a higher temperature can be carried out first, followed by tasks where the temperature may be lower. For example, the sequence of tasks optimizes the use of the resource.

According to the present disclosure, the determination of the operating parameters may be carried out taking into account a system configuration of the dialysis system, in particular the relative arrangement of the components and/or the hydraulic connections between the components.

For example, the operating parameters can be determined taking into account a sequence in which the components are hydraulically connected or connectable, in particular using existing hydraulic connecting elements and/or using switching elements between the components.

According to the present disclosure, determining the operating parameters may comprise time synchronization of the component operating data.

The time synchronization of the component operating data can include synchronizing the operating data of different components to a unified time and using the synchronized operating data to determine the operating parameters. For example, operating parameters can be provided for coordinated operation. For example, the synchronization can provide a clear relative temporal classification of the operating data. The method makes it possible to operate components with their respective system time and independently of the system time of the other components, while still enabling time coordination.

According to the present disclosure, determining the operating parameters may include detecting target conflicts for operating the dialysis system based on component operating data and/or resource availability data, and/or task prioritization data, and initiating automatic conflict resolution and/or initiating a user output indicating a target conflict.

Target conflicts can include, for example, that available resources, e.g. determined from the resource availability data, are not sufficient to execute all tasks according to the component operating data and/or task prioritization data. In such a case, automatic, e.g. rule-based, conflict resolution can be initiated. For example, a specific target conflict can be identified, an associated rule can be selected from a plurality of rules that specify how the target conflict is to be resolved for potential target conflicts, and the target conflict can be resolved in accordance with this rule. Alternatively or additionally, a user output, for example a message on a screen or an acoustic or visual warning signal, can be triggered to indicate the target conflict. The user can then take steps to resolve the conflict.

According to the present disclosure, the method may comprise receiving, by the control device, data from at least one external information system and determining the operating parameters taking into account the data received from the external information system.

The external information system can be considered as an information system that is not part of the dialysis system. For example, it may be a hospital information system (HIS) or an information system that provides component specifications. In particular, the external information system can be located spatially separated from the dialysis system, especially outside a dialysis center. The data can be received via standard data interfaces.

It is possible, for example, to obtain information from a HIS or a component manufacturer information system that is relevant to the operation of at least one of the components. Taking such data into account can improve the selection of operating parameters. For example, such data can provide information about possible operating modes or margins in the operating parameters, so that the operating parameters can be optimized for the benefit of resource consumption.

According to the present disclosure, the method may comprise an exchange of data, initiated by the control device, between at least two of the components indirectly via the control device.

For example, the control device can cause data that the control device has received from one of the components to be transmitted to another of the components. The exchange of data between two components initiated by the control device can be unidirectional or bidirectional. The exchange of data initiated by the control device can take the form of multicasting the data received by the control device from one of the components to several of the other components. In particular, the data may be component operating data. For example, a switch-on, a switch-over, a switch-off and/or a malfunction of a component can be reported to the control device and, via the control device, indirectly to one or more other components.

According to the present disclosure, the resource consumption may comprise an energy consumption and/or a water consumption and/or a time consumption.

The energy consumption can, for example, be the amount of energy required to complete the task. The energy consumption can include, for example, the consumption of electrical energy and/or energy in the form of heat. Water consumption can be, for example, the amount of water required to complete the task. The water consumption may be the consumption of a certain type of water, for example treated water. The time requirement can be, for example, the time required to complete the task. Other conceivable resources are ingredients to be added to the water, for example electrolytes, or liquids other than water.

According to the present disclosure, at least some of the components can be only hydraulically connected to each other. In particular, these components may not have a direct data connection. In other words, these components can only exchange data indirectly via the control device. Direct data exchange between these components may therefore not be possible. This can ensure improved security and it is also not necessary to provide compatible interfaces and protocols.

According to the present disclosure, the components may comprise at least one reverse osmosis device and/or at least one concentrate mixing system and/or at least one hot cleaning system and/or at least one dialysis machine.

The reverse osmosis device can be configured for (dialysis) water treatment. The reverse osmosis device can provide treated water as a resource. In addition to time, the resource consumption of the reverse osmosis device can include water consumption, for example of used water, and energy consumption.

The concentrate mixing system can be configured for mixing (dialysis) concentrate. The concentrate mixing system can provide dialysis concentrate as a resource. The resource consumption of the concentrate mixing system may include, in addition to time, a water consumption, an energy consumption, including electrical energy and heat, and an additive. Temperature plays a role in how quickly and how well the concentrate is mixed.

The hot cleaning system can be configured for hot cleaning containers and pipes of the dialysis system and for providing hot water for disinfecting the dialysis equipment. Furthermore, the hot cleaning system can provide heated water for dissolving powder concentrates in concentrate mixing systems. The resource consumption of the hot cleaning system may include, in addition to time, water consumption and energy consumption, including electrical energy and heat.

The dialysis machine may be configured to perform dialysis on a patient. The resource consumption of the dialysis machine may include, in addition to time, a consumption of dialysis concentrate, a consumption of water and an energy consumption, including electrical energy and heat. The dialysis machine may provide used water as a resource.

As a result, the various components compete for resources and sometimes provide resources that other components may be able to use instead of accessing external resources. It is therefore apparent that coordinated operation of the components can improve resource consumption and that it is possible to optimize operating parameters based on information about upcoming tasks and related requirements, priorities of tasks and resources.

In particular, the method of the present disclosure can be carried out completely automatically, in particular by the control device, unless explicitly stated otherwise.

The present disclosure also provides a dialysis system comprising a plurality of hydraulically connected components and a control device, wherein the control device is connected to each of the components by means of a data connection, wherein the dialysis system is configured to perform the method according to the present disclosure.

In particular, the data connection can be a data connection that enables bi-directional data exchange, i.e. in particular transmission of data from the control device to the component and from the component to the control device.

The present disclosure also provides a system comprising the dialysis system.

The system can also comprise an information system external to the dialysis system, for example a hospital information system (HIS) or an information system that provides component specifications. In particular, the external information system may be spatially separate from the dialysis system, especially outside a dialysis center.

With regard to the other features and advantages, reference is made to the explanations above.

The present disclosure also provides a computer program product comprising instructions that cause the dialysis system of the present disclosure to perform the method steps according to the present disclosure, in particular as described above.

The present disclosure also provides a computer-readable medium on which the computer program product of the present disclosure is stored.

The features and advantages described in connection with the method also apply mutatis mutandis to the dialysis system, system, computer program product and computer-readable medium.

### BRIEF DESCRIPTION OF THE DRAWINGS

Further examples and embodiments are explained below with reference to the figures, of which:

FIG. 1 shows a schematic representation of a system according to the present disclosure;

FIG. 2 shows a schematic representation of a method according to the present disclosure;

FIG. 3 shows a schematic representation of a system according to the present disclosure; and

FIG. 4 shows a schematic representation of a system according to the present disclosure.

### DETAILED DESCRIPTION

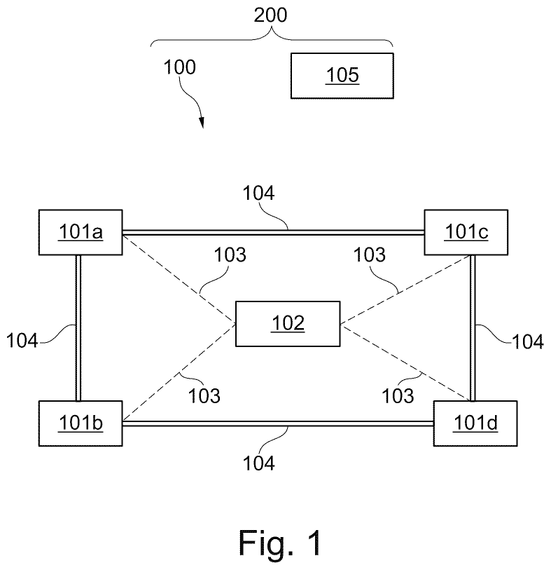

FIG. 1 shows a system 200 according to the present disclosure comprising a dialysis system 100 according to the present disclosure with a plurality of hydraulically connected components 101a, 101b, 101c, 101d and a control device 102, wherein the control device is connected to each of the components by means of a data connection 103, e.g. for bi-directional data exchange. The hydraulic connection is identified by the reference sign 104. In the present example, the components are not connected to each other via a data connection. For example, they can be connected to each other exclusively hydraulically.

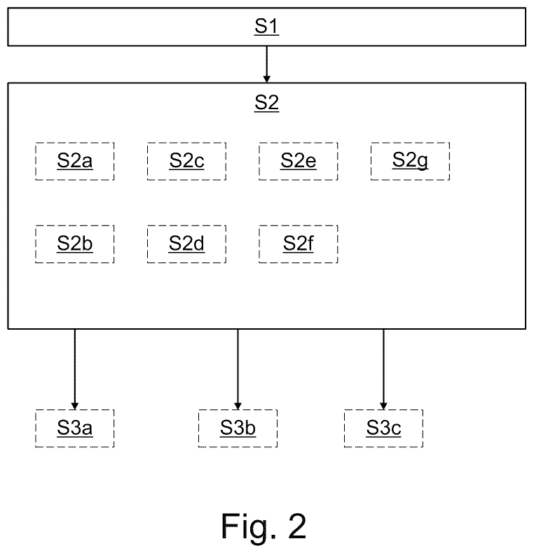

The system shown in FIG. 1 and described above can be configured to carry out the method according to the present disclosure, in particular also the method steps described below in connection with FIGS. 2 to 4. In particular, the control device can be configured to carry out or control at least some of the method steps, in particular all method steps.

The system can optionally also comprise an information system 105 external to the dialysis system, for example a hospital information system (HIS) or an information system that provides component specifications. In particular, the external information system may be spatially separated from the dialysis system, in particular outside a dialysis center.

FIG. 2 schematically illustrates a method for operating a dialysis system comprising several hydraulically connected components, for example medical devices, and a control device, according to the present disclosure.

The method comprises, in step S1, receiving component operating data from at least one of the components by the control device. The component operating data comprises at least demand data indicating an expected resource consumption of the component for an upcoming task to be performed by the component. The resource consumption may include, for example, an energy consumption and/or a water consumption and/or a time consumption.

The method comprises, in step S2, determining, by the control device, operating parameters for coordinated operation of the components such that an expected resource consumption of the dialysis system is optimized.

Determining the operating parameters comprises determining, in step S2a, at least one operating parameter for one or more of the components based on the component operating data and based on resource availability data and/or task prioritization data.

Optionally, determining the operating parameters may comprise, in step S2b, grouping requirements of the components and/or prioritizing the tasks to be performed. Optionally, determining the operating parameters may comprise, in step S2c, determining a timing of the execution of the tasks. Optionally, determining the operating parameters may comprise, in step S2d, time synchronization of the component operating data.

Optionally, determining the operating parameters may comprise, in step S2e, detecting target conflicts for operating the dialysis system based on component operating data and/or resource availability data and/or task prioritization data. Determining the operating parameters may further optionally comprise, in step S2f, initiating automatic conflict resolution and/or, in step S2g, causing a user output indicating a target conflict.

In the present method, the operating parameters can be determined taking into account the availability of resources, in particular taking into account the availability of energy and/or the availability of fluids.

Alternatively or in addition, in the present method, the operating parameters can be determined taking into account a system configuration of the dialysis system, in particular the relative arrangement of the components and/or the hydraulic connections between the components.

The operating parameters may optionally also be determined taking into account data received from an external information system. The method can optionally comprise receiving this data in step S3a.

The method may comprise that, in optional step S3b, the control device receives measurement data from one or more components of the dialysis system on the inflowing fluid volume and the outflowing fluid volume. This data can be used to determine the operating parameters. In particular, balancing can be performed based on this data.

The method may comprise, in optional step S3c, an exchange of data, initiated by the control device, between at least two of the components indirectly via the control device. This data can be used to determine the operating parameters.

Further embodiments and advantages of methods or systems according to the present disclosure are described below.

With current procedures and systems, a time window for a planned action is typically programmed into each individual medical device of a dialysis system. This results in various problems.

One example relates to inline hot cleaning in the dialysis system. The heating capacity of an inline hot cleaning system is limited by the maximum heating capacity of the heaters and can only supply a limited number of dialysis machines with hot water at the same time. If too many dialysis machines draw hot water from the ring line at the same time, the water temperature required for thermal disinfection is no longer reached as the heating capacity of the hot cleaning system is exceeded.

This also applies to concentrate mixing systems (CMS) in the dialysis system. These require reverse osmosis water to dissolve the concentrates in batch form. When supplying the concentrate mixer, reverse osmosis water preheated by the hot cleaning process may not be provided to speed up the mixing of the dialysis concentrate. This makes mixing less efficient.

In addition, current processes and systems do not allow leakage monitoring during water withdrawal, as the withdrawal quantities of the consumers are not known.

In general, current procedures and systems do not automatically prioritize tasks in the dialysis system. In many dialysis centers, for example, the safe disinfection of dialysis machines before use on the patient has the highest priority, before the disinfection of the ring line, before the disinfection of the reverse osmosis and ultimately also before the mixing of dialysis concentrate, as this can also be brought to the treatment site in canisters.

There is also no bundling of tasks in current processes and systems. However, this can be advantageous, as explained below. For example, although dialysis machines can be supplied with hot water in blocks (of five, for example), all machines can be rinsed out simultaneously with cold water when the ring line cools down (to save energy). As a further example, the A0 value of 600 for the ring line can also be achieved during DI of the dialysis machines without a separate heating window. Thermal disinfection with moist heat in washer-disinfectors is defined and controlled parametrically via the A0 value in accordance with the EN ISO 15883-1 standard (cleaning and disinfecting devices-Part 1: General requirements, definitions and tests). The A0 value is the measurement of the energy used (temperature/time), which shows whether the disinfection process has achieved the desired lethality or not. “A” is the time equivalent in seconds at 80° C. An A0 value of 600 (e.g. 10 min/80° C. or 1 min/90° C.) is considered sufficient for the disinfection of bacteria and fungi, but also covers a number of thermolabile viruses and noroviruses.

This allows for saving energy and time. During hot water withdrawal from the dialysis machines, the continuous application of hot water to the ring line also automatically disinfects the ring line.

Also, with current procedures and systems, clocks of the medical devices (MDs) operated together often do not run synchronously, which makes coordinated operation difficult, for example. It is also difficult to synchronize error logs. When switching between summer and winter time, this can also lead to errors when running programs synchronously.

In summary, with current procedures and systems, manual programming of medical devices that are only hydraulically connected to each other leads to a waste of resources, namely water, energy and/or time, as well as other problems.

The method and system of the present disclosure allow at least some of the above problems to be solved.

According to one example of the present disclosure, all components (medical devices) in a dialysis center that are only hydraulically connected to each other, for example reverse osmosis, concentrate mixing systems, hot cleaning systems, dialysis machines, are to be coordinated in their operating modes during the operating time by a central control system (for example by the control device) in such a way that the consumption of resources (energy, water) can be optimized, operating times can be minimized and, optionally, leakages can be reliably detected.

All hydraulically connected devices can register their requirements with the central control system, for example for dialysis-free periods.

The central control system can group and prioritize the requests in such a way that all requests are carried out according to their importance, the consumers are switched on in such a way that resource consumption is optimized and unresolvable conflicts of objectives are reported, the sequence of controlled activities is logged, energy consumption is aligned with energy availability (example: controlled batch disinfection of consumers taking into account the available heating capacity), and the transmission of operating data and settings to one or more network participants enables efficient operation.

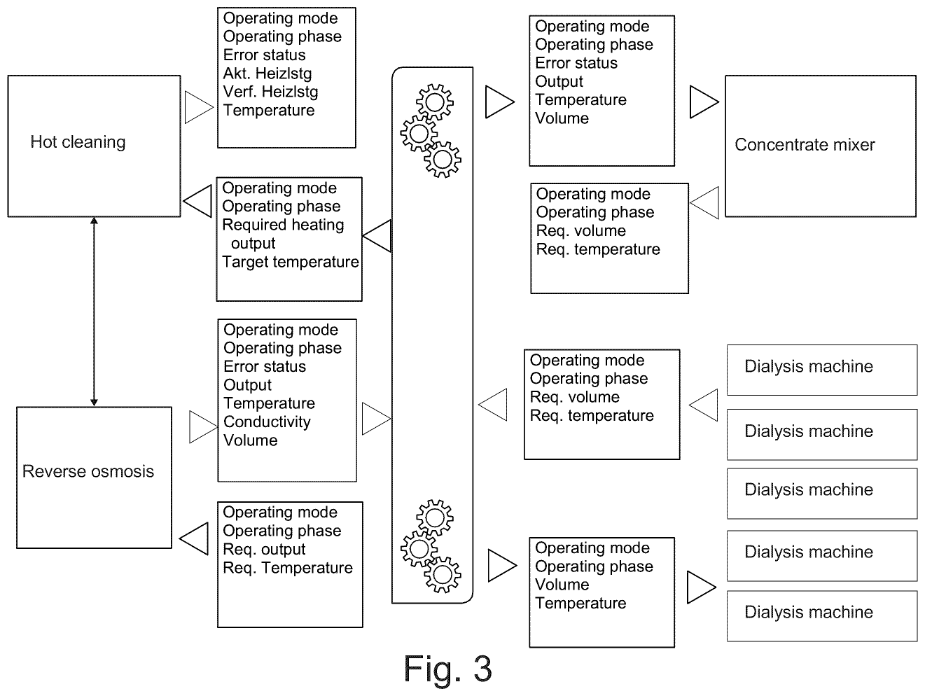

FIG. 3 shows a schematic representation of the possible connected active components/medical devices in a dialysis system according to the present disclosure. The information pointing to the central controller is provided by the connected components/medical devices. The information pointing out from the central controller is provided to components by the central controller.

In particular, the components “hot cleaning”, “reverse osmosis”, “concentrate mixer” and “dialysis machine” are shown here as examples.

The components can each report the operating mode and operating phase to the control unit. In addition, error status, current heating power (e.g. in kW), available heating power (e.g. in kW) and temperature can also be reported by the hot cleaning system. Error status, output (e.g. in liters/hour), temperature, conductivity and volume can also be reported by the reverse osmosis. The concentrate mixer and the dialysis machines can also report volume requirements (“Req. volume”), for example water from the reverse osmosis, and temperature requirements (“Req. temperature”), for example the temperature of the water coming from the reverse osmosis.

The central control system can then report the operating mode, operating phase, error status, output, temperature, and volume to the components, for example. In addition, the required heat output and target temperature can be reported to the hot cleaning and/or volume requirement (“Req. volume”) and temperature requirement (“Req. temperature”) to the reverse osmosis.

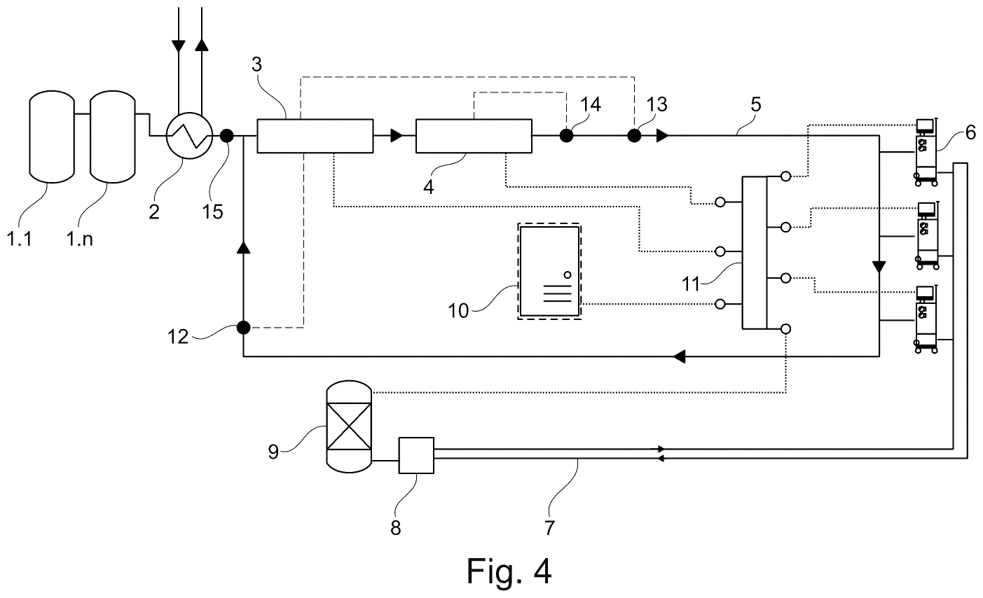

FIG. 4 shows a schematic representation of the hydraulic and information technology connections in an exemplary dialysis center comprising an exemplary dialysis system according to the present disclosure.

In the figure, the dialysis system comprises the following exemplary components:
1.1 Pre-treatment unit 1
1.n Pre-treatment unit n
2 Heat exchanger
3 Reverse osmosis
4 Hot cleaning
Permeate ring line
6 Consumer/dialysis machine
7 Concentrate ring line
8 Concentrate distribution system
9 Concentrate mixer
Control system
11 Network
12 Flow meter outlet permeate ring line
13 Flow meter inlet permeate ring line
14 Temperature sensor for hot cleaning
15 Temperature sensor outlet heat exchanger

None of the above-mentioned elements, with the exception of the control unit and any suitable data connections, is mandatory. Subgroups of the above elements can be suitably combined with each other. In particular, the dialysis system can be configured without a concentrate mixer 9. The dialysis system can also be configured without upstream connection of the heat exchanger and without the temperature sensor outlet heat exchanger 15.

In the following, an exemplary method according to the present disclosure is described. In describing the method, reference is made only by way of example to the system shown in FIG. 4. However, the method can also be carried out using other suitable systems.

According to the method, all components (medical devices) connected to a control unit 10 via a network 11 can register their requirements, for example volume, temperature, operating mode and/or operating phase, centrally with the control unit. After determining the resource requirements, for example permeate volume requirements, temperature requirements and/or time requirements, of all connected components/medical devices, the controller can, for example, determine an optimum sequence for connecting all consumers.

For example, it can be determined so that the maximum heating capacity of the hot cleaning system 4 is not exceeded at any time.

Alternatively or additionally, the determination can be made so that program steps in hot disinfection processes that do not require hot water (in particular the rinsing of the dialysis machines 6 after chemothermal disinfection) can be carried out with cold water.

Alternatively or additionally, the determination can be carried out so that the process step “hot cleaning of the ring line” is covered by an A0 value calculation of the disinfection performance achieved when supplying the dialysis machines with hot water. This takes into account that the provision of hot water for the disinfection of the dialysis machines simultaneously heats up the ring line so that the ring line is disinfected at the same time as the dialysis machines are disinfected.

Alternatively or additionally, the determination can be made so that the concentrate mixing system 9 is fed with water at a predetermined temperature (see sensor 14), which is suitable for accelerating the dissolving process of the salt. The ready-mixed concentrate is then conveyed to the dialysis machine 6 with the aid of a concentrate distribution system 8 and a concentrate ring line 7.

For example, the network can send a target temperature for the feed water 16 to the heat exchanger 2 so that the reverse osmosis membranes are operated with low transmembrane pressure and thus, in addition to reducing the reheating (see above), the electrical power consumption of the high-pressure pumps of the reverse osmoses is reduced.

Optionally, leakage monitoring can be enabled by balancing the volume flows at the inlet 13 and outlet 12 of the permeate ring line 5 based on the registered requirements of the connected consumers.

Although the present disclosure is illustrated and described in detail in the figures and the foregoing description, these figures and descriptions are to be considered exemplary and not limiting. The present disclosure is not limited to the embodiments shown. In view of the foregoing description and figures, it will be apparent to those skilled in the art that various modifications can be made within the scope of this disclosure.

1. A method of operating a dialysis system comprising a plurality of components that are hydraulically connected and a control device, the method comprising:
receiving, by the control device, component operating data from at least one component of the plurality of components, the component operating data comprising at least demand data indicating an expected resource consumption of the at least one component for an upcoming task to be performed by the at least one component; and
determining, by the control device, operating parameters for coordinated operation of the plurality of components in such a way that an expected resource consumption of the dialysis system is optimized,
wherein determining the operating parameters comprises determining, based on the component operating data and based on resource availability data and/or task prioritization data, at least one operating parameter for one or more of the plurality of components.

2. The method according to claim 1, wherein determining the operating parameters comprises a grouping of requirements of the plurality of components and/or a prioritizing of tasks to be performed.

3. The method according to claim 1, wherein determining the operating parameters comprises determining a timing of execution of tasks.

4. The method according to claim 1, wherein determination of the operating parameters is carried out taking into account resource availability.

5. The method according to claim 1, wherein determination of the operating parameters is carried out taking into account a system configuration of the dialysis system.

6. The method according to claim 1, wherein determining the operating parameters comprises time synchronization of the component operating data.

7. The method according to claim 1, wherein determining the operating parameters comprises detecting target conflicts for operating the dialysis system based on component operating data and/or resource availability data, and/or task prioritization data, and initiating automatic conflict resolution and/or initiating a user output indicating a target conflict.

8. The method according to claim 1, wherein the method comprises receiving, by the control device, data from at least one external information system and determining the operating parameters taking into account the data received from the at least one external information system.

9. The method according to claim 1, comprising an exchange of data, initiated by the control device, between at least two components of the plurality of components indirectly via the control device.

10. The method according to claim 1, wherein the expected resource consumption comprises an energy consumption and/or a water consumption and/or a time consumption.

11. The method according to claim 1, wherein at least some components of the plurality of components are only hydraulically connected to one another.

12. The method according to claim 1, wherein the plurality of components comprise at least one reverse osmosis device and/or at least one concentrate mixing system and/or at least one hot cleaning system and/or at least one dialysis machine.

13. A dialysis system configured for carrying out the method according to claim 1, the dialysis system comprising a plurality of hydraulically connected components and a control device, the control device being connected to the plurality of hydraulically connected components by a respective data connection.

14. A computer program product comprising instructions that cause a dialysis system to perform the steps of claim 1.

15. A computer-readable medium on which the computer program product according to claim 14 is stored.
